(19) United States
(12) Patent Application Publication
(10) Pub. No.: US 2006/0252918 A1
(43) Pub. Date: Nov. 9, 2006

(54) Use of hepatitis virus (hcv) p7 protein

(76) Inventors: David Rowlands, Leeds (GB); Shawn Cannon Lemon, Leeds (GB); Syephen Griffin, Leeds (GB)

Correspondence Address: MYERS BIGEL SIBLEY & SAJOVEC, PO BOX 37428, RALEIGH, NC, 27627, US

(21) Appl. No.: 10/520,550

(22) PCT Filed: Jul. 7, 2003
(86) PCT No.: PCT/GB03/02937

§ 371 Date: May 17, 2006

(30) Foreign Application Priority Data
Jul. 8, 2002 (GB) 0215617.2

(51) Int. Cl. C12Q 1/70; C07K 14/18

(52) U.S. Cl. 530/350; 435/5

(57) ABSTRACT

The present invention relates to the use of hepatitis C virus (HCV) p7 protein, and particularly but not exclusively, to its use in rationalised drug design and as a screen for antiviral therapeutic agents.

The present invention relates to the use of hepatitis C virus HCV) p7 protein, and particularly but not exclusively, to its use in rationalised drug design and a method therefor and also it its use in a screen for antiviral therapeutic agents.

### BACKGROUND TO THE INVENTION

Hepatitis C virus (HCV) is the prototype member of the Hepacivirinae genus of the Flaviviridae. The viral genome is a single coding sense RNA of around 9.5 Kilobases and encodes a single polyprotein of around 3000 amino acids translated in a cap-independent manner from an Internal Ribosome Entry Site (IRES). The polyprotein contains the viral structural proteins towards the N-terminus, and the non-structural replicative proteins in the C-terminal two thirds of the molecule. Individual proteins are generated from this precursor by the action of both host and viral proteases. Replication of HCV RNA is thought to occur in the cytoplasm of the infected cell in complexes associated with cellular membranes derived from the Endoplasmic Reticulum (ER), leading to the generation of new viral progeny which are released through the secretory pathway.

The HCV viral polypeptide comprises 10 viral proteins in the order of: NH(2)-Core-E1-E2-p7-NS2-NS3-NS4A-NS4B-NS5A-NS5B-COOH

Located at the junction between the viral structural and non-structural genes, the p7 protein of HCV is a 63 amino acid protein of a highly hydrophobic nature (accession number AF054247 (HCVJ4)). Sequence analysis suggests that p7 forms an integral membrane protein with two alpha-helical trans-membrane domains of an amphipathic nature separated by a short stretch of charged residues. p7 has been shown to localise to the ER and plasma membrane and is predicted to have its' termini present in the ER lumen and the charged region on the cytosolic side from CONFIRMATION COPY topological studies. However, the function of HCV p7 in the virus life cycle is not known.

Hepatitis C virus (HCV) infection has emerged as the major cause of non-A, non-B viral hepatitis (NANBH) in the world. Current estimates from the World Health Organisation predict that over 3% of the world population are currently infected with the virus, making it a major public health issue in many countries. Exposure to HCV via contact with infected blood leads in most cases to a chronic persistent infection of the liver. Furthermore, this process is often asymptomatic thereby delaying clinical intervention until late stage disease manifests in the form of liver cirrhosis, often leading to end stage liver failure or hepatocellular carcinoma; a rapidly progressive cancer with a poor prognosis. Current treatment of HCV disease comprises type I Interferon often in combination with Ribavirin, there being no vaccine currently available. However, this treatment is often ineffective against the HCV genotypes common in the USA and Western Europe, and therefore there is a need for new and effective anti-viral agents/therapies.

### STATEMENT OF THE INVENTION

The present invention resides in the surprising observation that HCVp7 forms ion channels both in vitro and in vivo (in hepatocyte-derived cell lines) thus making it a suitable target for rationalised drug design of anti-viral compounds.

As used herein the word “comprises” is not exclusive, i.e. it indicates that the subject of the verb need not consist only of its object but may include the object of the verb and one or more additional elements. Cognate expressions are to be construed accordingly.

According to a first aspect of the invention there is provided use of HCVp7, a variant, functionally effective fragment or a mutation thereof that retains ion channel forming capability in screening candidate compounds that inhibit or increase ion channel activity.

Reference herein to an HCVp7 variant, functionally effective fragment or a mutation thereof is intended to include any part of the sequence identified as accession number AF054247 (HCVJ4) or its expression products which has ion channel activity.

Preferably, HCVp7 is coupled to a poly(amino acid) sequence.

Coulping may be for example by covalent bonding, homo or heterofunctional linking or through chemical cross-linkage or by a natural pepetide.

Preferably, the poly(amino acid) sequence comprising basic natural or unnatural amino acids such as ARG, LYS or HIS.

Preferably, the linker is a poly HIS comprising at least 2 and up to 50 residues.

Preferably, the poly HIS comprises at least 2 and up to 15, or at least 2 and up to 10 or more preferably still at least 2 and up to 6 and preferably at least 4 residues.

Preferably, the HCVp7 is incorporated into a membrane for example and without limitation a black lipid membrane.

In another embodiment of the invention nucleic acid encoding the HCVp7 protein, variant, functionally effective fragment or a mutation thereof is incorporated into or comprised in a viral system.

Reference herein to viral system is intended to include, examples such as and without limitation a herpes virus, adenovirus, pestivirus such as bovine viral diarrhoea virus, picomavirus, Flavivirus or pox virus vector.

According to a yet further aspect of the invention there is provided a method of screening a compound, preferably from a compound library for compounds, that inhibit or enhance ion channel activity comprising the steps of:
(i) contacting a membrane comprising an HCVp7 protein or a viral system including a nucleic acid encoding an HCVp7 protein with a candidate compound; and
(ii) measuring ion channel activity across said membrane or in viral system.

According to a yet further aspect of the invention there is provided a method of screening a compound or a compound library for efficacy of inhibition or enhanced ion channel activity comprising the steps of:
(i) contacting a membrane comprising a HCVp7 protein with a candidate compound or a viral system including a nucleic acid encoding an HCVp7 protein with a candidate compound; and
(ii) comparing the activity of said candidate compound with a standard.

The standard may be a known inhibitor or enhancer of ion channel activity, for example amantadine.

It will be appreciated that the methods of the present invention advantageously allow for high-throughput screening of large drug libraries for compounds that inhibit or increase ion channel activity or compounds with improved efficacy over prior art compounds.

Preferably, the methods of the invention further include any one or more of the preferred features hereinbefore disclosed.

In one embodiment of the invention the method may comprise combining amantadine therapy with another antiviral compound or amantadine may for comparative purposes.

According to a yet farther aspect of the invention there is provided use of HCVp7 in the assessment of channel formation by p7 variants and mutants thereof.

According to a yet further aspect of the invention there is provided a compound identified according to the method of the invention.

According to a yet further aspect of the invention there is provided an antiviral therapeutic agent as identified by the method of the invention.

According to a yet further aspect of the invention there is provided use of a therapeutic agent identified by the method of the present invention in the preparation of a medicament for the treatment of a viral infection.

According to a yet further aspect of the invention there is provided use of a therapeutic agent identified by the method of the present invention in the preparation of a medicament for the treatment of hepatitis.

According to a yet further aspect of the invention there is provided use of a therapeutic agent identified by the method of the present invention in the preparation of a medicament for the treatment of hepatitis C virus (HCV) infection.

According to a yet further aspect of the invention there is provided use of an antibody directed against HCVp7 as an inhibitor of channel ion activity, pharmaceutical preparations thereof and use in the manufacture of a medicament for the treatment of hepatitis C virus (HCV) infection.

According to a yet further aspect of the invention there is provided a membrane incorporating HCVp7, a variant, functionally effective fragment or a mutation thereof that retains ion channel forming capability. The membrane may be used in the method or for the uses hereinbefore described in any of the other aspects of the present invention.

The invention will now be described by way of example only with reference to the following Figures wherein:

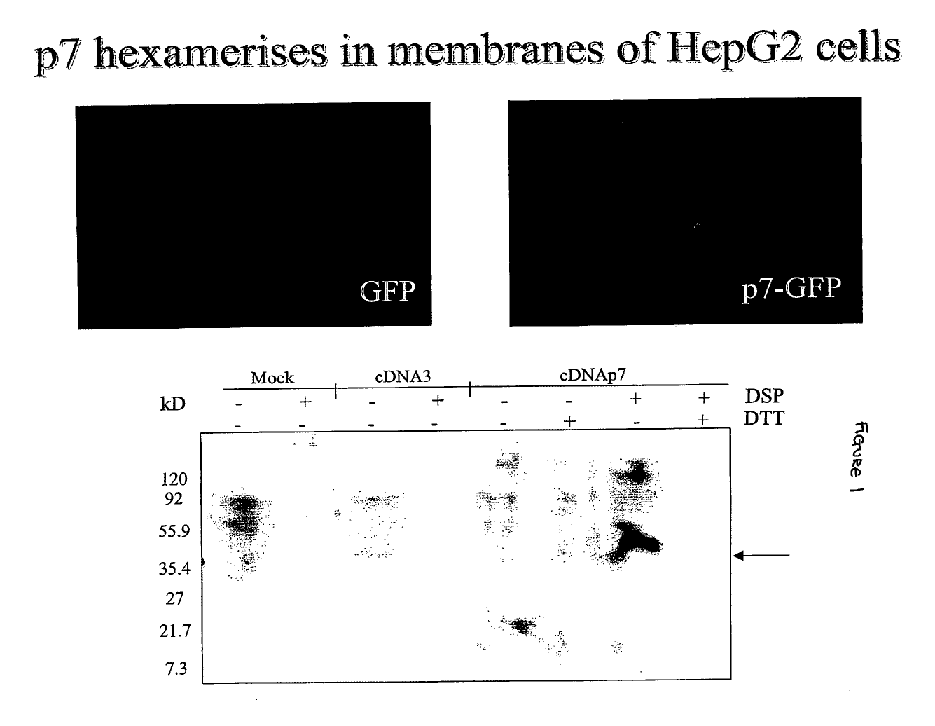

FIG. 1 illustrates p7 hexamerisation in membranes of HepG2 cells;

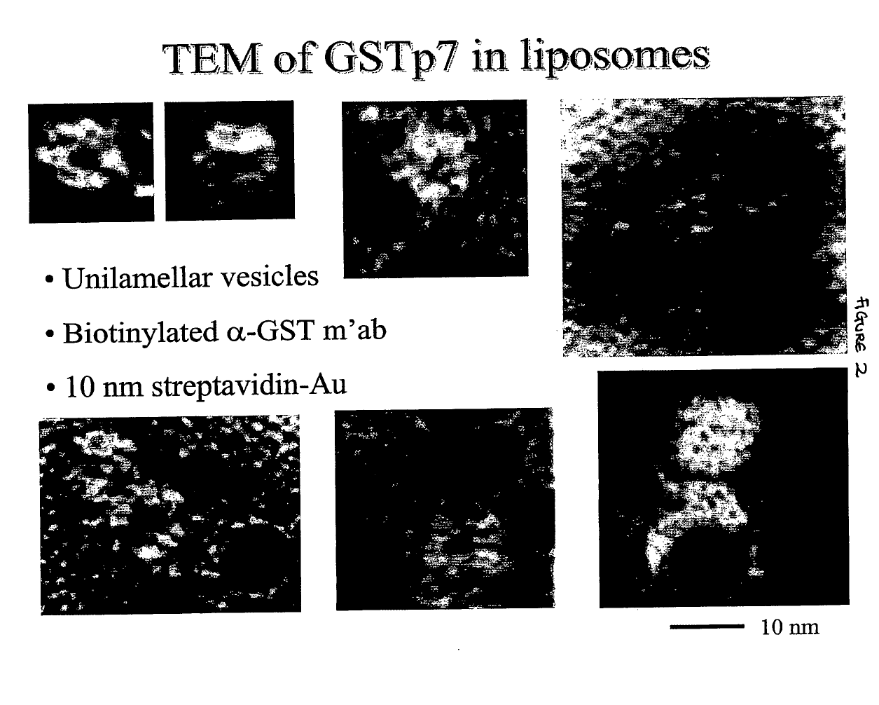

FIG. 2 shows transmission electron microscope images of GSTp7 in liposomes;

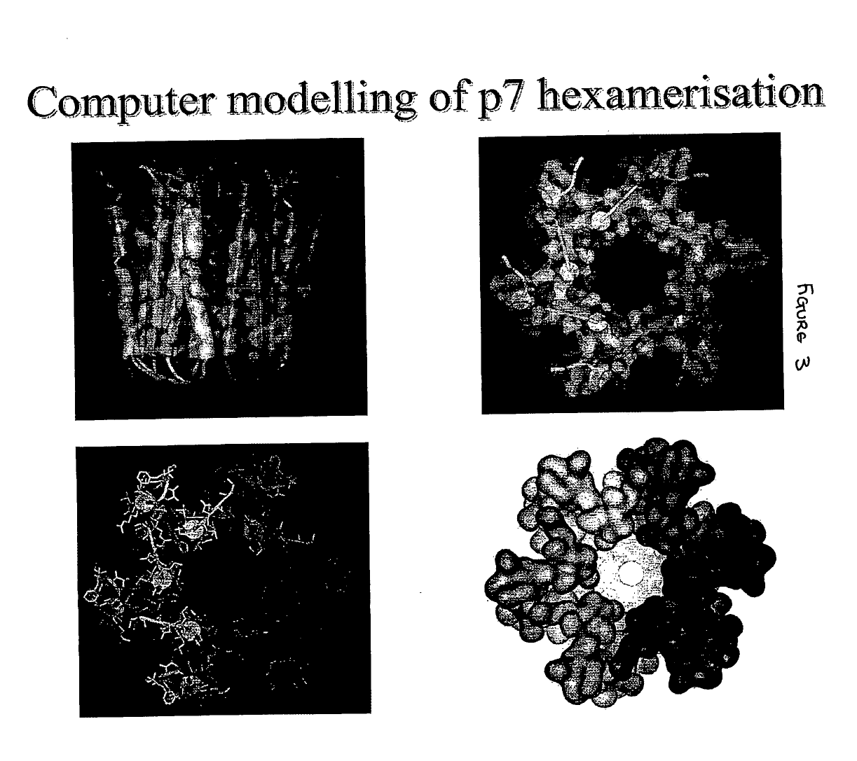

FIG. 3 illustrates computer modelling of p7 hexamerisation;

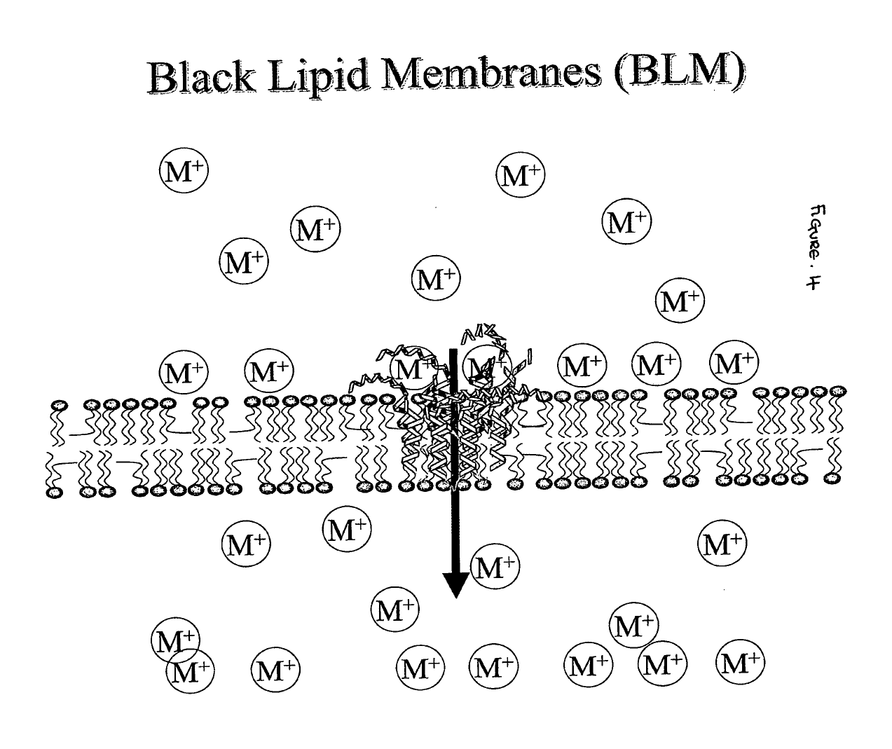

FIG. 4 a schematic representation of a black lipid membrane (BLM);

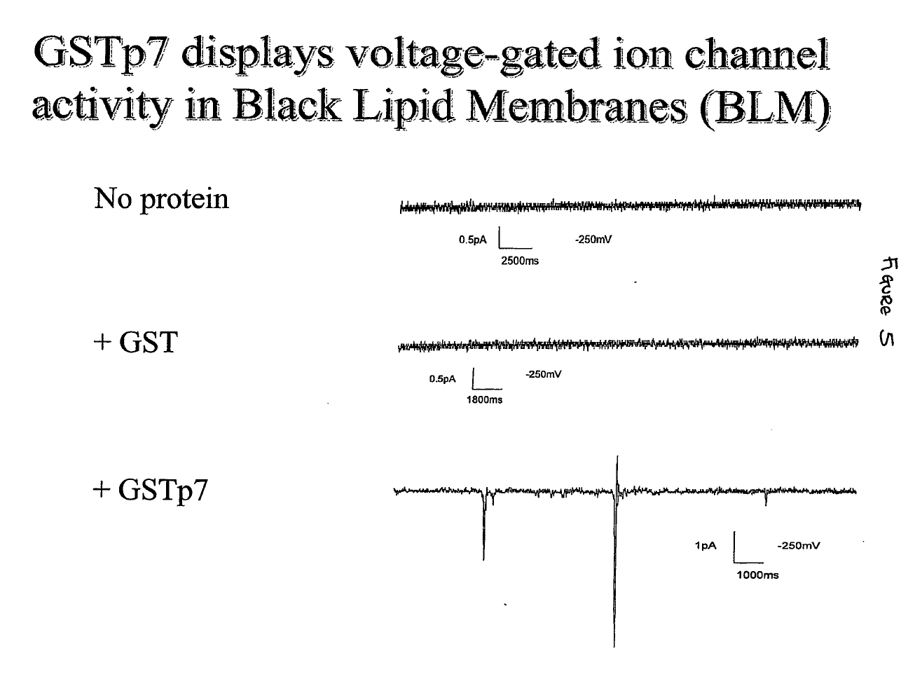

FIG. 5 shows GSTp7 voltage-gated ion channel activity in BLMs;

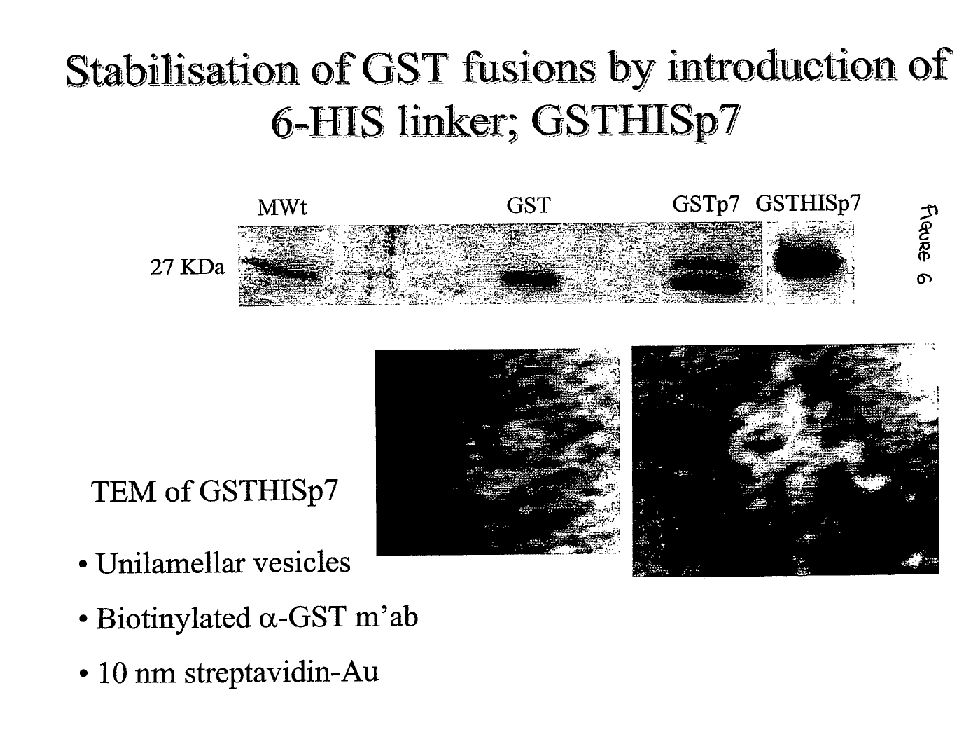

FIG. 6 shows GSTHISp7 stabilisation;

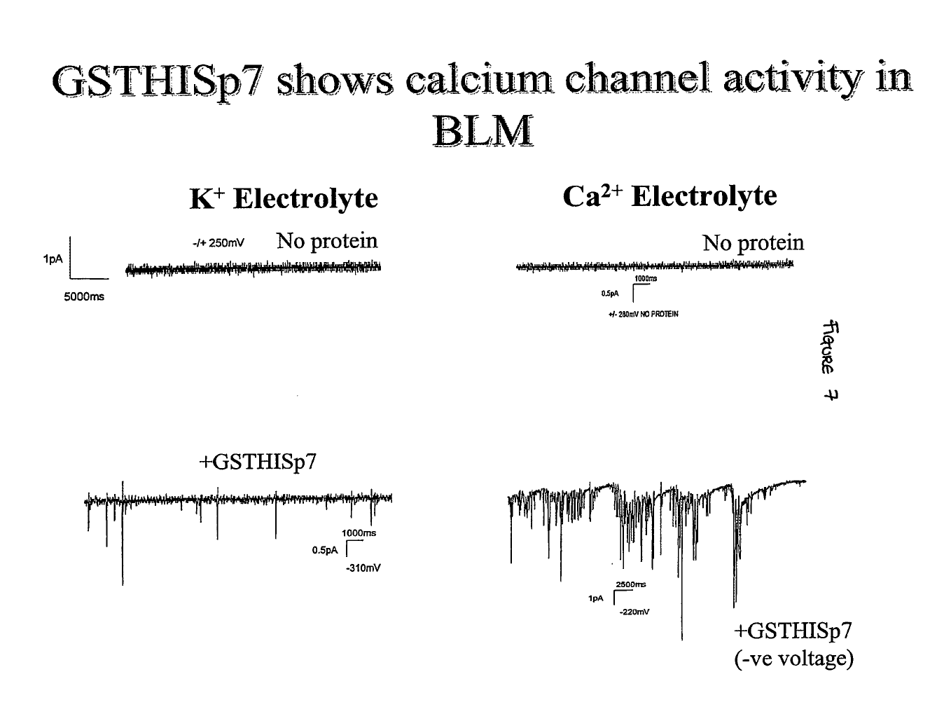

FIG. 7 shows GSTMSp7 calcium ion channel activity;

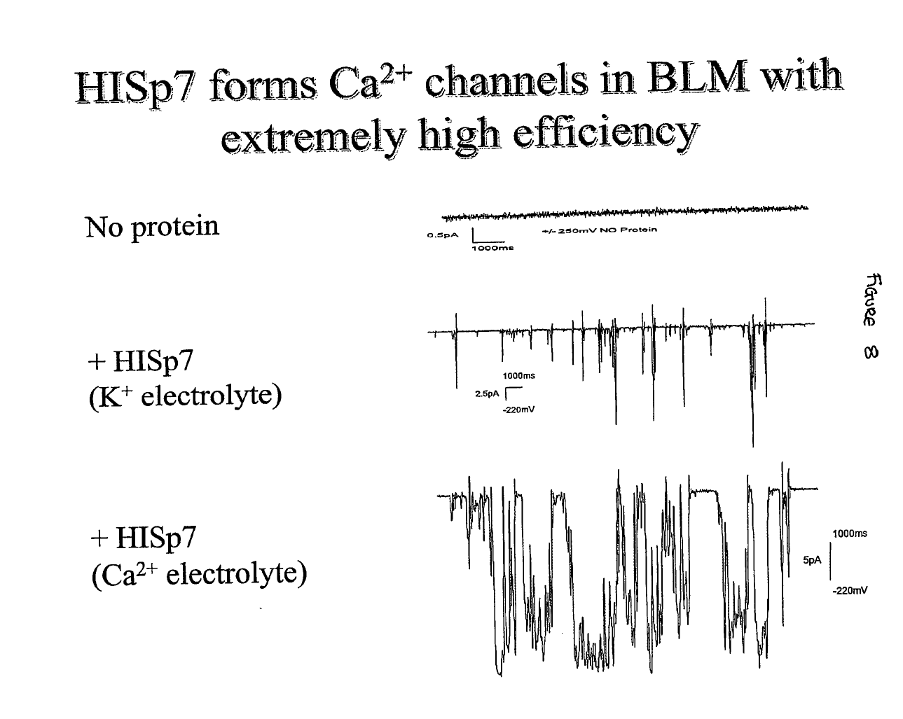

FIG. 8 shows HISp7 calcium ion channel activity;

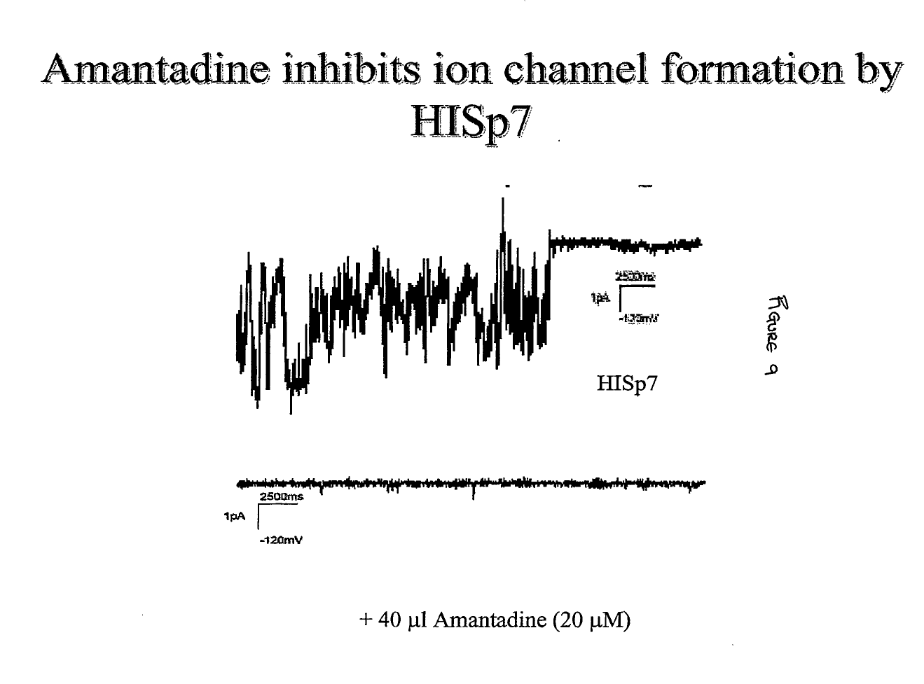

FIG. 9 shows amantadine inhibits HISp7 ion channel formation;

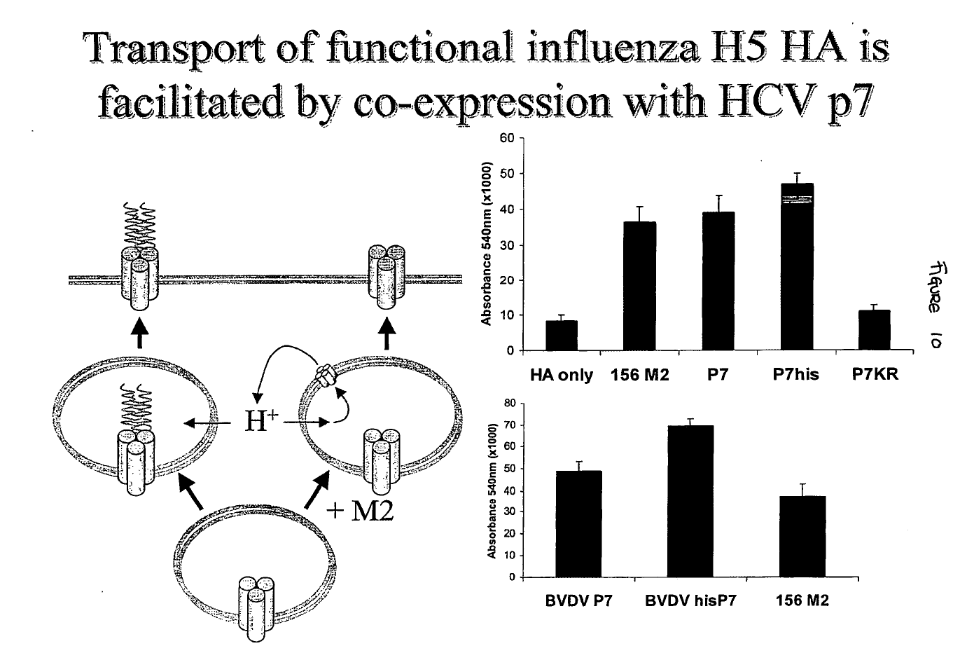

FIG. 10 shows the putative method of transport of functional influenza H5 HA and facilitation by co-expression with HCVp7 and;

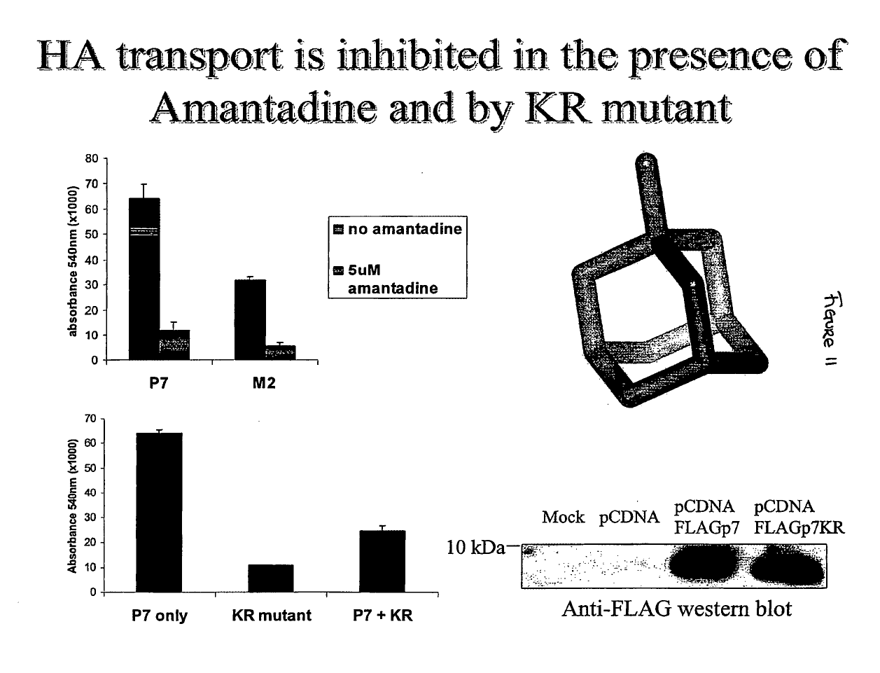

FIG. 11 shows HA transport is inhibited in the presence of amantadine and by KR mutant.

### DETAILED DESCRIPTION OF THE INVENTION

Many animal viruses encode proteins of low molecular weight, which are hydrophobic and form oligomers. When these proteins are individually expressed in bacteria or in animal cells, they induce profound modifications in cellular permeability. These proteins therefore, have been collectively termed as “viroporins”. Amongst the viral proteins that enhance membrane permeability are poliovirus 2B, 2BC and 3A, the togavirus 6K polypeptide, influenza M2 and Vpu from HIV-1. These Viroporins are all small integral membrane proteins that oligomerise to form ion channels in cellular and often viral membranes. They usually function so as to modulate cation exchange to facilitate egress of virus particles from cells or changes to the interior of virus particles. Perhaps the most famous of these proteins is the M2 protein of Influenza A virus which is the target of the first anti-viral drug; Amantadine. We provide evidence that p7 is a Viroporin and that it too will oligomerise in membranes to form ion channels in a similar fashion as M2 thus making HCVp7 a suitable target for anti-viral compounds.

Materials and Methods

BLM Experimental Procedures

Solutions/Chamber Preparation

All buffer solutions were prepared by dissolving the relevant amounts of KCl, CaCl₂ (Both Aldrich 99+%) and PBS in Millipore water (≧18 MΩ) to give the following concentrations. 0.1M, 0.2M, 0.5M, 1M and 4M. A commercially available BLM chamber was pre-cleaned by immersion in DECON/Millipore water (≧18 MΩ) for 24 hrs prior to all experiments. To remove all traces of detergent the chamber was flushed with running water for at least five hours. Immediately before use the chamber was washed extensively with Millipore water (≧18 MΩ) and dried in N₂. Silver chloride electrodes were prepared using electrochemical deposition of chloride onto silver wire (d=1 mm) from a concentrated KCl solution. Agar bridges were prepared by cleaning glass pipettes in Methanol (HPLC grade) then storing in a drying oven. The pipettes were moulded into the correct shape using glass blowing techniques. A 4M buffer solution containing 2% bacterial agar was pipetted and the Agar bridges thus formed were stored in 4M buffer solution until required.

Lipid Preparation

A number of lipid compositions were investigated using the following methodology. A 30 μl aliquot of phosphatidylethanolamine (25 mg ml⁻¹—Lipid Products) was added to 38 μl of phosphatidylserine (25 mg ml⁻¹—Lipid Products). The solvent was removed with N₂ and the lipids were dried under vacuum for 3 hours. After drying the lipids were redissolved in 30 μl decane (Aldrich 99.5+%), vortexing as required, then stored on ice prior to use.

Lipid Bilayer Formation/Recording

The two Ag/AgCl electrodes were placed in a Faraday cage to minimise noise during current recordings and connected to a computer via an AXON patchclamp filtered at 50 Hz, an ADC interface and a DAT recorder. AXON pclamp software was utilised to record and analyse the traces. A sample of the lipid in decane solution was brushed around the chamber cup pore (200 μm) to act as a “glue” and aid stable bilayer formation. The chambers were filled with the required buffer solution and the current and capacitance monitored to ensure that the cup pore was unblocked. A sample of the lipid solution was brushed across the cup pore until a stable capacitance was recorded. The lipid was then allowed to thin and stabilise over a 15 min period. Only membranes that gave zero current and specific capacitances of 0.3-1μF cm⁻² were used further for protein studies. The cis chamber was clamped and the trans chamber applied voltage was varied between +/−280 mV to monitor the stability of the bilayer and to determine the presence of possible contaminants.

Protein/Amantadine Studies

Varying amounts (15-100 μl) of the proteins under study (GST, GSTp7, GSThisp7 and hisp7 in methanol or PBS— see detailed description of the invention) were injected into the trans compartment of the BLM chamber. After 10 minutes the applied voltage was varied between +/−280 mV and the resultant current signals recorded as a function of time.

To monitor the effect of amantadine (Aldrich) on the formation of ion channels, 401 μl of amantadine (20 μM in methanol) was added to both cis and trans compartments.

The current traces showing blocking of ion channels were recorded within 30 secs after amantadine injection.

Detailed Protocol for Cloning/Expression/Purification of GSTp7, GSTHISp7, and HISp7.

Generation of plasmid constructs. The p7 sequence of hepatitis C virus 1B was amplified via PCR using the J4 isolate infectious clone pCVJ46LS as a template (Virology. 1998 Apr. 25;244(1):161-72). PCR was carried out using a proof-reading thermostable polymerase; Vent polymerase (New England Biolabs) according to manufacturers instructions. The p7 cassette was generated using primers; newp7Fwd 5′-ATATATGAATTCGCGGCCATGGCCTTAGAGAACTTGGTG-3′ (SEQ ID NO:1) and newp7Rev 5′-ATATATACTGCAGGCGGCCGCGGCGTAAGCTCG TGGTGGTAACG-3′ (SEQ ID NO:2). The HISp7 cassette was generated using primers; newp7Rev (above), and HISp7Fwd 5′-ATATATGAATTCGCGGCCAT GCATCATCATCATCATCATGCCTTAGA GAAC TTG-3′ (SEQ ID NO:3). PCR amplified DNA was extracted with phenol/chloroform (25:1) pH 8.0, ethanol precipitated, and digested with Eco RI and Not I restriction endonucleases (New England Biolabs) at 37° C. for 3 hours. Resulting sticky-ended DNAs were purified by agarose gel electrophoresis followed by phenol extraction and ligated to the Glutathione-S-Transferase expression vector, pGEX4T1 (Amersham Pharmacia Biotech, Genbank accession number U13853) which had been digested and purified in the same manner, using a rapid DNA ligation kit (Roche Diagnostics). Ligations were transformed into E. coli DH5α and resulting clones were confirmed by restriction digest to release the cloned fragment and by double stranded DNA sequencing (Lark Technologies, UK). Plasmids were named pGEXp7 and pGEXHISp7.

Expression and purification of GSTp7. A single colony from a fresh transformation of pGEXp7 was used to inoculate a 5 ml overnight culture (LB+100 μg/ml Ampicillin) grown at 30° C. This was then used to seed a 400 ml culture which was grown at 30° C. to an OD₆₀₀ of 1.0. At this point, IPTG (Isopropyl β-D-thiogalactopyranoside) was added to a final concentration of 0.1 mM in order to induce expression from the Taq promoter, and the cultures grown for a further 2 hours. Cells were pelleted at 6000 rpm in a Sorvall SLA-3000 rotor for 10 min at 4° C. The resulting pellet was resuspended in 10 ml PBS containing 1 mM DTT (Dithiothreitol) and protease inhibitor cocktail (Roche Diagnostics). 0.5 ml of lysozyme (10 mg 1 ml) was then added and the mixture incubated at room temperature for 5 min to clear. Large cellular debris was disrupted by sonication, followed by the addition of 1 ml PBS/DTT/10% Triton X-100 and centrifugated (Sorvall SLA-1500 rotor) at 10000 rpm for 10 min to pellet debris. 1 ml of a 1:1 suspension of glutathione-sepharose beads was then added to the supernatant and the mixture rotated at 4° C. for 1 h. Beads were then washed three times in PBS/DTT/protease inhibitor, and finally resuspended in PBS/DTT at a 1:1 ratio v/v. Beads were loaded onto a gravity column (Clontech) and washed three times with 50 mM Tris-Cl, pH 8.0 to equilibrate. Fusion proteins were then eluted by the addition of 3×0.5 ml Tris-Cl, pH 8.0 containing 20 mM reduced Glutathione (SIGMA). The second and third elutions were pooled and dialysed using a Slide-a-lyzer cassette (Pierce Endogen) in PBS or MeOH. Purity and concentration of the protein was then determined by SDS-PAGE and BCA.

Expression and purification of GSTHISp7. GSTHISp7 was expressed and purified in the same way as GSTp7, except that instead of a starter culture, the 400 ml culture was inoculated with a single colony and grown for 12 h at 30° C. before induction with 0.1 mM final concentration IPTG, followed by growth overnight at the same temperature.

Generation of HISp7 from GSTHISp7 by thrombin cleavage. Pre-dialysis, GSTHISp7 was cleaved at the thrombin cleavage site present in the pGEX4T1 polylinker by the addition of 10 units/mg fusion protein thrombin (SIGMA). Incubation was carried out overnight at room temperature and the cleaved HISp7 separated by GS-trap™ (Amersham Pharmacia Biotech) chromatography followed by collecting the flow-through after passing through a 10 000 MWt filter (Microsep, Pall life sciences). Purity and concentration were then determined by mass spectometry, SDS-PAGE and BCA.

Haemadsorption Assay

Vero cells were prepared to about 70% confluency in 6-well trays and then incubated overnight at 37° C. Cells were washed once in PBS and 1 ml of a 1:10 dilution of T7 (diluted in serum-free medium) was added to each well. This was then incubated at 37° C. for a further 1 hr and washed once in PBS. The transfection mix (see below). Was then added and incubated for 5-12 h at 37° C., the mix was removed and 2 ml of medium with 10% FCS was added with a further incubatation period of 48 h at 37° C. Untransfected control and infected positive control were also prepared, the positive was infected with virus 24 h after the transfection.

The bacterial mixture was then diluted to a concentration of 5.5mU/ml with medium (1:182 dilution), 1 ml of sample was added to each well and incubated at 37° C. for 1 h and then washed three times with PBS. 1 ml of 0.5% horse red blood cells was added to each well and incubate for at least 2 hr at room temperature. Plates were agitated to re-suspend all loose red blood cells and washed gently three times with PBS. 1 ml of 1× CAT lysis buffer was added to each well and left for 1 minute to lyse the cells. Samples were then microfuged at 13,000 rpm for 3 min and the supernatant decanted into a plastic cuvette so that the absorbance could be read at 540 nm.

Lipofectamine Transfection

DNA was made up to 100 μl with optimem in a bijou bottle and 4 μl lipofectamine added to 96 μl optimem in another bijou bottle. (41 per 1 μg DNA and 1 μg of HA and 0.2 μg of M2 were used). The DNA mix was then added to the lipofectamine mix and incubated for 30-45 min at room temperature. Vero cells were then washed with serum-free medium, and 800 μl of optimem added to each transfection mix. The mix was then dripped onto the cells.

### EXAMPLE 1

As previously discussed, Viroporins are all small integral membrane proteins that oligomerise to form ion channels in cellular and often viral membranes. They usually function so as to modulate cation exchange and to facilitate egress of virus particles from cells or changes to the interior of virus particles.

With reference to FIGS. 1 and 2 it has been shown that HCVp7 forms hexamers both in vitro in HePG2 cells and in vivo in liposomes. FIG. 3 illustrates the computer modelling of HCVp7 hexaherisation. These observations coupled with the hydrophobic nature at the amino acid level suggest that HCVp7 is indeed a member of the Viroporin family. FIG. 4 provides a schematic representation of HCVp7 incorporated in a BLM.

### EXAMPLE 2

With reference to FIG. 5, we have been able to demonstrate that a GSTp7 fusion protein has voltage-gated ion channel activity in BLM. Moreover, stability of the fusion protein increases by the incorporation of a 6-HIS linker as seen in FIG. 6. In addition, we have been able to demonstrate that the inclusion of a 6-HIS linker increases ion channel activity in the presence of both K⁺ and Ca²⁺ electolytes as seen in FIG. 7. The effect being more pronounced in the presence of the Ca²⁺ electolyte.

### EXAMPLE 3

We have found that removal of the GST part of the fusion protein, so that p7 is associated only with the 6-HIS linker, resulted in an unexpected 5 fold increase in ion channel activity in the presence of both K⁺ and Ca²⁺ electolytes (FIG. 8). The ion channel activity still being more pronounced in the presence of the Ca²⁺ electolyte. These results are surprising since p7, which has two a helices, is lipid soluble and was fused to GST in order to make the molecule more soluble. Accordingly these results suggest that HISp7 acts as a voltage-gated calcium channel BLM in the absence of a fusion protein and that it represents a novel target for screening compounds that inhibit ion channel activity.

### EXAMPLE 4

Our studies have demonstrated that amantadine inhibits ion channel formation by HISp7 FIG. 9) in the micromolar range. This confirms the potential use of HISp7 as a target for screening inhibition of channel activity and may lead to the discovery of alternative anti-viral therapies.

### EXAMPLE 5

Using the haemadsorption assay and Vero cells we have been able to show that transport of functional influenza H5 HA is facilitated by co-expression with HCV p7 (FIG. 10) and that HA transport is inhibited by the presence of amantidine and by the K33A/R35A mutation (FIG. 11). We believe that HA flu protein is shipped to the cell surface where it adopts a fusogenic state (see schematic representation). However, the presence of either M2 or p7 prevents HA from becoming fusogenic so that it is able to bind to sialic acid on red blood cells. We have also shown that the his-tag does not substantially alter activity and that expression (as demonstrated by Western blot FIG. 11) is not affected by the presence of the his-tag and that the KR mutation is dominant negative and that mutation does not affect expression. We have also been able to demonstrate that p7 ion channel activity is substantially abrogated in the KR mutant and that bovine viral diarrhoea virus (BVDV) p7 also mediates mammalian cell membrane permeability (FIG. 10). These data support the present invention that p7 forms ion channels and has utility in the pharmaceutical industry.

Our studies have shown that we are able to express the p7 protein of HCV alone or as part of a fusion protein in vitro, in bacteria and mammalian hepatocyte-derived cell lines. We have observed by electron microscopy a hexameric form of p7 fusion proteins purified from bacteria and the frequency of this oligomeric form is greatly enhanced in the presence of lipid membranes. The hexameric form is entirely attributable to the presence of the p7 domain as none was seen in preparations of the fusion protein partner alone. Furthermore, following expression of p7 alone in hepatocyte-derived cells a 42 KDa species was detected by western blotting. This species was only detected in gels run under denaturing conditions after prior stabilisation with a lipid-soluble chemical cross-linking reagent, suggesting that its formation occurred within cellular membranes. These properties are characteristic of viroporins, which mediate cation permeability across membranes and are important for viral particle release or maturation. We believe that p7 is of particular utility as a target for rationalised drug design of antiviral therapies and that including p7 in a membrane will offer an improved screening system and method for detecting candidate therapeutics.

1. Use of HCVp7, a variant, functionally effective fragment or a mutation thereof in screening candidate compounds that inhibit or increase ion channel activity.

2. Use according to claim 1 wherein HCVp7 is coupled to a poly(amino acid) sequence.

3. Use according to claim 2 wherein the poly(amino acid) sequence linker comprises a basic natural amino acid selected from the group consisting of ARG, LYS and HIS.

4. Use according to claim 2 wherein the poly(amino acid) sequence is a polyHIS sequence.

5. Use according to claim 4 wherein the polyHIS sequence comprises at least 2 and up to 50 residues.

6. Use according to claim 5 wherein the polyHIS sequence comprises at least 2 and up to 10 residues

7. Use according to claim 1 wherein HCVp7 is incorporated into or comprised in a membrane.

8. Use according to claim 7 wherein the membrane is a black lipid membrane.

9. Use according to claim 1 wherein a nucleic acid encoding the HCVp7 protein, variant, functionally effective fragment or a mutation thereof is incorporated into or comprised in a viral system.

10. A method of screening for compounds that inhibit or enhance ion channel activity comprising the steps of:
(i) contacting a membrane comprising a HCVp7 protein or a viral system comprising a nucleic acid encoding an HCVp7 protein with a candidate compound; and
(ii) measuring ion channel activity across said membrane or in a viral system.

11. A method of screening a compound for efficacy of inhibition or enhanced ion channel activity comprising the steps of:
(i) contacting a membrane comprising a HCVp7 protein or a viral system comprising a nucleic acid encoding an HCVp7 protein with a candidate compound; and
(ii) comparing the activity of said candidate compound with a standard.

12. (canceled)

13. Use of HCVp7 in the assessment of ion channel formation by p7 variants and/or mutants thereof.

14. (canceled)

15. A compound identified according to the method of claim 10.

16. An antiviral therapeutic agent as identified by the method of claim 10.

17. Use of a therapeutic agent identified by the method of claim 10 in the preparation of a medicament for the treatment of a viral infection.

18. Use of a therapeutic agent identified by the method of claim 10 in the preparation of a medicament for the treatment of hepatitis.

19. Use of a therapeutic agent identified by the method of claim 10 in the preparation of a medicament for the treatment of hepatitis C virus (HCV) infection.

20. Use of an antibody directed against HCVp7 as an inhibitor of channel ion activity, pharmaceutical preparations thereof and use therefor in the manufacture of a medicament for the treatment of hepatitis C virus (HCV) infection.

21. A membrane incorporating HCVp7, a variant, functionally effective fragment or a mutation thereof that retains ion channel forming capability.

22. (canceled)

23. Use of a membrane according to claim 21 in screening candidate compounds that inhibit or increase ion channel activity.

24. Use of a membrane according to claim 21 in the method of claim 10.

25. A compound identified according to the method of claim 11.

26. An antiviral therapeutic agent as identified by the method of claim 11.

27. Use of a therapeutic agent identified by the method of claim 11 in the preparation of a medicament for the treatment of a viral infection.

28. Use of a therapeutic agent identified by the method of claim 11 in the preparation of a medicament for the treatment of hepatitis.

29. Use of a therapeutic agent identified by the method of claim 11 in the preparation of a medicament for the treatment of hepatitis C virus (HCV) infection.

30. Use of a membrane according to claim 21 in the method of claim 11.


SEQUENCE LISTING (3 sequences)

SEQ ID NO: 1 - DNA, 39 bp, Artificial
  PCR primer
atatatgaattcgcggccatggccttagagaacttggtg

SEQ ID NO: 2 - DNA, 33 bp, Artificial
  PCR primer
atatatactgcaggcggccgcggcgtaagctcg

SEQ ID NO: 3 - DNA, 54 bp, Artificial
  PCR primer
atatatgaattcgcggccatgcatcatcatcatcatcatgccttagagaacttg